(19) United States
(12) Patent Application Publication
(10) Pub. No.: US 2024/0299423 A1
(43) Pub. Date: Sep. 12, 2024

(54) COMPOSITIONS FOR TREATMENT OF DIABETIC SYMPTOMS

(71) Applicant: Innocan Pharma Ltd., Herzliya (IL)

(72) Inventor: Nir AVRAM, Metar (IL)

(73) Assignee: Innocan Pharma Ltd., Herzliya (IL)

(21) Appl. No.: 18/574,027

(22) PCT Filed: Jul. 6, 2022
(86) PCT No.: PCT/IL2022/050728

§ 371 Date: Dec. 24, 2023

Related U.S. Application Data
(60) Provisional application No. 63/219,500, filed on Jul. 8, 2021.

(51) Int. Cl. A61K 31/00; A61K 8/34; A61K 8/37; A61K 8/49; A61K 8/891; A61K 8/92; A61K 9/00; A61K 9/06; A61K 31/165; A61K 36/23; A61K 36/25; A61K 36/31; A61Q 19/00

(52) U.S. Cl. CPC A61K 31/658; A61K 8/342; A61K 8/345; A61K 8/375; A61K 8/4946; A61K 8/891; A61K 8/92; A61K 9/0019; A61K 9/06; A61K 31/165; A61K 36/23; A61K 36/25; A61K 36/31; A61Q 19/007

(57) ABSTRACT

Described herein are compositions which can be used to improve circulation and thereby treat ailments of diabetic patients. The compositions comprise a cannabinoid, preferably cannabidiol. Further described herein are compositions comprising a cannabinoid, and a blood flow enhancing agent. Methods for treatment of diabetic patients are also disclosed.

### CROSS REFERENCE TO RELATED APPLICATIONS

Benefit is claimed to U.S. Provisional Patent Application No. 63/219,500 filed Jul. 8, 2021; the contents of which are incorporated by reference herein in its entirety.

### FIELD

Provided herein are compositions comprising cannabinoids for treatment of conditions including conditions associated with diabetes.

### BACKGROUND

Diabetes is a condition associated with the body's production or use of insulin. In healthy individuals, insulin is produced in the pancreas and is instrumental in regulating the individual's blood sugar (glucose) levels. In diabetic patients, the body does not produce enough insulin or the body does not react to the insulin produced, impacting the body's ability to regulate glucose levels. In some diabetic patients, the faulty regulation of glucose can cause various ailments such as, but not limited to: heart disease, vision impairment, skin conditions, and kidney disease.

It has been found that diabetes can lead to poor blood circulation, especially in the body's extremities, for example, the feet. Poor blood circulation attributed to diabetes may stem from high glucose levels in the blood damaging blood vessels, making it difficult for the blood vessels to provide blood to the neighboring cells, in particular, in the periphery. It is suggested that diabetics have regular checks of their feet, including testing circulation in the feet. Poor circulation in diabetics may lead to conditions, including skin conditions and infections. In extreme situations, diabetics having poor circulation in their feet may require a part or an entire foot to be amputated.

### SUMMARY

Described herein are compositions which can be used to improve circulation and thereby treat ailments of diabetic patients. The compositions comprise a cannabinoid, preferably cannabidiol.

Further described herein are compositions comprising a cannabinoid, and a blood flow enhancing agent. Methods for treatment of diabetic patients, particularly via the topical route, using these compositions are described herein.

The foregoing and other objects, features, and advantages will become more apparent from the following detailed description.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

### Definitions

Brassica alba Sprout Extract: An extract form the sprouts of the plant Brassica alba, also known as mustard.

Cannabinoid: A cannabinoid is a chemical compound that acts on cannabinoid receptors in cells in mammals, including in humans. Cannabinoids can be manufactured synthetically or obtained from various parts of the genus Cannabis, in particular, from the species Cannabis Sativa. Cannabinoids from the cannabis plant are referred to as phytocannabinoids. Two preferred cannabinoids according to various embodiments, are (−)-trans-Δ⁹-tetrahydrocannabinol, and/or isomers thereof (THC) and cannabidiol (CBD). Alternatively, a cannabinoid may be in the form of cannabis extract. Alternatively, a cannabinoid may be in the form of a synthetic cannabinoid. Compositions described herein may comprise one cannabinoid or multiple cannabinoids, such as a combination of CBD and THC. Other cannabinoids which can be used in compositions described herein include but are not limited to one or a combination of: cannabigerol (CBG), cannabigerolic acid (CBGA), cannabigerol monomethyl ether (CBGM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C₄ (CBN-C₄), cannabinol C₂ (CBN-C₂), cannabinol C₁ (CBN-C₁), cannabinodiol (CBND), cannabinovarinic acid (CBNVA), cannabinovarin (CBNV), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran (DCBF), cannabirispol (CBR).

Capsaicin: An organic compound produced by plants of the genus Capsicum, and having the structure:

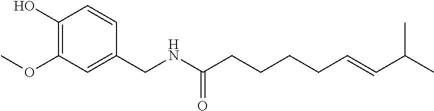 [SMILES: COc1cc(CNC(=O)CCCC/C=C/C(C)C)ccc1O]

### DESCRIPTION OF EMBODIMENTS

Described herein, according to some embodiments, are compositions comprising a cannabinoid, for administration to a patient suffering from a diabetic ailment. The compositions may further comprise a blood flow enhancing agent. Preferably, a blood flow enhancing agent is an agent, when applied topically, increases blood flow in the patient's body in a local area after application. Exemplary blood flow enhancing agents include caffeine, methyl nicotinate, ginger root oil, Brassica alba Sprout Extract, capsaicin, Hedera helix (Ivy) Leaf Extract, Centella asiatica Extract, Fucus vesiculosus Extract, Vitis vinifera (Grape) Leaf Extract, Alpha Glucosyl Hesperidin, Aesculus hippocastanum (Horse Chestnut) Seed Extract, Cupressus sempervirens Fruit Extract, Vaccinium myrtillus Fruit Extract, Papaver rhoeas Petal Extract, Thymus serpyllum Extract, Ruscus aculeatus Root Extract, Adiantum Capillus-veneris Leaf Extract, Thymus serpyllum Extract, Phospholipids (and) Escin (and) Beta-Sitosterol, Xymenynic Acid, Vanillyl Butyl Ether, Ruscus aculeatus Root Extract (and) Citrus limon (Lemon) Peel Extract (and) Solidago virgaurea (Goldenrod) Extract (and) Astragalus membranaceus Root Extract.

Compositions described herein are preferably administered through the topical route and may be formulated as a cream, an ointment, an emulsion, a stick, a spray, a roll-on or a moisturizing gel. Optionally, the spray may be a continuous spray, comprising a propellant. Optionally, the spray may be a bag on valve spray.

The amount of cannabinoid may range from between 0.05% by weight to 1% by weight, preferably between 0.1% to 0.2% by weight.

The amount of blood flow enhancing agent may range from between 0.001% and 10% by weight.

The amount of Brassica alba sprout extract may range from between 0.001% by weight to 0.01% by weight, preferably 0.008% by weight.

The amount of capsaicin may range from between 0.001% by weight to 0.1% by weight, preferably 0.01% weight.

The amount of caffeine may range between 0.1% and 1% weight by weight, preferably between 0.2% and 0.5% by weight.

The amount of methyl nicotinate may range between 0.1% and 2% by weight.

A single cannabinoid or a mixture of cannabinoids, such as a cannabis extract, comprising multiple cannabinoids may be used in compositions described herein. Optionally, the cannabinoid used is cannabidiol.

The composition may further comprise a skin protectant. Optionally, the skin protectants is one, or more than one of the following: allantoin, beeswax, calamine, calcium acetate, cetyl alcohol, cocoa butter, colloidal oatmeal, dexpanthenol, dimethicone, glycerin, glyceryl stearate, kaolin, lanolin, mineral oil, petrolatum, zinc acetate, and zinc oxide. The skin protectant preferably is a silicone oil such as dimethicone. Preferably, the skin protectant is petrolatum. Preferably, the skin protectant is glycerin.

According to an embodiment, the composition comprises hemp oil in the oil phase of the composition. Optionally, hemp oil is present in the composition in an amount of between 0.1% and 5% of the composition. Optionally, hemp oil is present in an amount of 0.5% of the composition.

Optionally, an additional ingredient may be added. The ingredient may be selected from the group consisting of: urea, hydroxyethyl urea, shea butter, pro vitamin b5, l-arginine, tea tree oil, ammonium lactate, lactic acid, sodium lactate, petrolatum, glycerin, hyaluronic acid, calendula oil, chamomile ext., jojoba oil, beeswax, aloe vera gel, licorice, evening primrose oil, coconut oil, cholesterol ceramides, allantoin, omega-6 oils, squalane, zinc pca, safflower seed oil, dimethicone, avocado oil, and borage seed oil,

Optionally, the compositions are in the form of a cream. The cream may comprise water, preferably in an amount of 50% or more water by weight.

Optionally, the compositions are in the form of an ointment. The ointment may comprise petrolatum, preferably in an amount of 40% by weight, or more.

Described herein, according to some embodiments, are methods for treatment of diabetic related conditions using a cannabinoid, applied via the topical route to the skin of a patient in need thereof. Optionally, the cannabinoid is CBD.

Administration of the compositions described herein may be performed topically, by applying to the skin. The composition should be applied to the skin affected, preferably on a foot or leg or the hand or the arm of the patient in need thereof, on a daily basis, optionally, twice, three of four times daily.

The amount administered per application for a composition may vary from between 0.1 g to 15 g per administration.

Compositions described herein are designed to have an immediate effect, to provide moisture to dry skin, to alleviate pain and discomfort, and to improve blood flow.

It is suggested that compositions described herein are beneficial in treating diabetic related conditions. Optionally, the diabetic-related condition is selected from the group consisting of: cold feet or hands, numbness in hands or feet, hair loss, dry skin, slow healing of wounds, ulcers, a bacterial infection of the skin, a fungal infection of the skin, itching, acanthosis nigricans, diabetic dermopathy, necrobiosis lipoidica diabeticorum, cruptive xanthomatosis, digital sclerosis, gangrene, necrosis, disseminated granuloma annulare, trans-epidermal water loss, skin redness, skin scales, skin roughness, and pain. The compositions described herein may improve skin hydration, skin moisturization, skin redness, provide soothing and contribute to smooth skin in diabetic related conditions.

Cannabinoids, in particular CBD, may have a synergistic effect in the treatment of a diabetic-related condition when used in combination with blood flow enhancing agents as described herein.

According to an embodiment, described herein, is a method for treatment of a patient suffering from a diabetic related condition comprising administering to the patient a composition comprising a cannabinoid, applied via the topical route to the skin of a patient in need thereof. Optionally, the composition is in the form of a cream, an ointment, an emulsion, a stick, a spray, a roll-on or a moisturizing gel. Optionally, the cannabinoid is present from between 0.05% to 1% by weight. Optionally, the cannabinoid is present from between 0.1% to 0.2% by weight. Optionally, the composition further comprises a blood flow enhancing agent. Optionally, the blood flow enhancing agent is selected from the group consisting of: Ivy extract, Centella asiatica extract, caffeine, methyl nicotinate, ginger root oil, Brassica alba Sprout Extract, and capsaicin. Optionally, the blood flow enhancing agent is present in the composition in an amount between 0.001% and −10% by weight. Optionally, the composition further comprises a skin protectant. Optionally, the skin protectant is selected from the group consisting of: allantoin, beeswax, calamine, calcium acetate, cetyl alcohol, cocoa butter, colloidal oatmeal, dexpanthenol, dimethicone, glycerin, glyceryl stearate, kaolin, lanolin, mineral oil, petrolatum, zinc acetate, and zinc oxide. Optionally, the skin protectant is dimethicone. Optionally, the skin protectant is petrolatum. Optionally, the skin protectant is glycerin. Optionally, the cannabinoid is CBD. Optionally, the condition is selected from the group consisting of: cold feet or hands, numbness in hands or fect, hair loss, dry skin, slow healing of wounds, ulcers, a bacterial infection of the skin, a fungal infection of the skin, itching, acanthosis nigricans, diabetic dermopathy, necrobiosis lipoidica diabeticorum, eruptive xanthomatosis, digital sclerosis, gangrene, necrosis, disseminated granuloma annulare, trans-epidermal water loss, skin redness, skin scales, skin roughness, and pain. Optionally, the composition is administered to a foot of the patient. Optionally, the composition is administered between 1 and 4 times daily. Optionally, the composition is administered in an amount of between 0.1 g to 15 g per administration.

Further described herein is a method for enhancing blood flow in a patient suffering from diabetes comprising administering to the patient a composition comprising a cannabinoid, applied via the topical route to the skin. Optionally, the patient experiences an improvement in skin hydration, skin moisturization, skin redness, soothing and skin smoothness

Further described herein is a composition for treatment of a patient suffering from a diabetic related condition comprising a cannabinoid, applied via the topical route to the skin of a patient in need thereof. Optionally, the composition further comprises a blood flow enhancing agent.

The following examples are provided to illustrate certain particular features and/or embodiments. These examples should not be construed to limit the disclosure to the particular features or embodiments described.

### EXAMPLES

### Example 1

A cream for treatment of diabetes-related indications and having CBD is prepared using the ingredients in table 1:

TABLE 1

| | Amount | |
|---|---|---|
| | (weight/ | |
| Ingredient | weight) % | Function |
| Aqua | 59.65 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 5.60 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Coco-Caprylate | 2.00 | Light emollient fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |

The first 6 ingredients of the water phase are added to a main vessel and heated to 75° C. In a separate vessel the following 19 ingredients of the oily phase are combined and heated to 75° C., while making sure that all waxes and solids are completely dissolved

The oily phase is added to the water phase in the main vessel at 75° C. and homogenized for 10 min. After homogenization, cooling with moderate mixing begins. At 40° C. a solution of water and hydroxyethyl urea is added while mixing, then the Aloe Barbadensis Leaf Juice and Chamomilla Recutita (Matricaria) Flower Extract are added.

Mixing and cooling is continued to 35° C.

### Example 2

A cream for treatment of diabetes-related indications and having CBD is prepared using the ingredients in table 2:

TABLE 2

| | Amount | |
|---|---|---|
| | (weight/ | |
| Ingredient | weight) % | Function |
| Aqua | 58.25 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Ricinus Communis | 3.00 | Emollient |
| (Castor) Seed Oil | | |
| Butyrospermum Parkii | 2.00 | Emollient |
| (Shea) Butter | | |
| Glyceryl Stearate + | 3.00 | Emulsifier |
| Sodium Stearate | | |
| Cetyl Esters | 2.00 | Thickener |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing & calming |
| Steareth-21 | 1.40 | Emulsifier |
| Coco-Caprylate | 2.00 | Light emollient, fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizing & Humectant |
| Aloe Barbadensis Leaf | 0.50 | Soothing and calming |
| Juice (and) Sodium | | |
| Benzoate (and) Potassium | | |
| Sorbate | | |
| Glycerin (and) Water (and) | 0.20 | Soothing and calming |
| Chamomilla Recutita | | |
| (Matricaria) | | |
| Flower Extract (and) | | |
| Sodium Benzoate (and) | | |
| Potassium Sorbate | | |
| Capric/Caprylic | 1.98 | Emollient |
| Triglyceride | | |
| Brassica Alba Sprout | 0.008 | Active component |
| Extract | | |
| Capsaicin | 0.01 | Active component |

The first 6 ingredients of the water phase are added to a main vessel and heated to 75° C. In a separate vessel the following 20 ingredients of the oily phase are combined and heated to 75° C., while making sure that all waxes and solids are completely dissolved.

The oily phase is added to the water phase in the main vessel at 75° C. and homogenized for 10 min. After homogenization, cooling with moderate mixing begins. At 40° C. a solution of water and hydroxyethyl urea is added while mixing, then the Aloe Barbadensis Leaf Juice and Chamomilla recutita (Matricaria) Flower Extract are added.

In a separate vessel, an oily liquid comprising the last three ingredients is prepared and added slowly to the main vessel then the mixture is homogenized for an additional two minutes. Mixing and cooling is continued to 35° C.

### Example 3

An ointment for treatment of diabetes-related indications comprising CBD is prepared using the ingredients in table 3:

TABLE 3

| | Amount | |
|---|---|---|
| | (weight/ | |
| Ingredient | weight) % | Function |
| Aqua | 18.50 | Carrier |
| Triethanolamine | 2.50 | p.H adjestment |
| Glycerin | 4.00 | Skin Protectant |
| Dimethicone | 1.00 | Skin Protectant |
| Petrolatum | 30.00 | Skin Protectant/Carrier |
| Stearic Acid | 5.00 | Emulsifier |
| Mineral oil | 22.00 | Skin Protectant |
| Ricinus Communis (Castor) | 5.00 | Emollient |
| Seed Oil | | |
| Beeswax | 8.00 | Skin Protectant |
| Cocamide MEA | 1.00 | Thickeneing & p.H |
| | | adjustment |
| Bisabolol | 0.10 | Soothing |
| Cannabis Sativa Seed Oil | 0.50 | Solvent |
| BHT | 0.05 | Preservative |
| Cannabidiol | 0.15 | Active |
| Tocopheryl Acetate | 0.20 | Anti Oxidant |
| Capric/Caprylic | 1.98 | Emollient |
| Triglyceride | | |
| Brassica Alba Sprout | 0.008 | Active |
| Extract | | |
| Capsaicin | 0.01 | Active |

The first 3 ingredients of the water phase are added to a main vessel and heated to 75° C. In a separate vessel the following 12 ingredients of the oily phase are combined and heated to 75° C., while making sure that all waxes and solids are completely dissolved.

The oily phase is added to the water phase in the main vessel at 75° C. and homogenized for 10 min. After homogenization, cooling with moderate mixing begins.

In a separate vessel, an oily liquid comprising the last three ingredients is prepared and added slowly to the main vessel then the mixture is homogenized for an additional two minutes. Mixing and cooling is continued to 35° C.

### Example 4

An ointment for treatment of diabetes-related indications comprising CBD is prepared using the ingredients in table 4:

TABLE 4

| | Amount | |
|---|---|---|
| | (weight/ | |
| Ingredient | weight) % | Function |
| Mineral Oil | 35.00 | Carrier |
| Ricinus Communis (Castor) | 5.00 | Emollient |
| Seed Oil | | |
| Lanolin Alcohol | 5.00 | Skin Protectant |
| Petrolatum | 41.00 | Carrier |
| Microcrystalline Wax | 10.00 | Emollient and |
| | | structuring |
| Bisabolol | 0.10 | Soothing & Calming |
| Cannabis Sativa Seed Oil | 0.50 | Solvent |
| BHT | 0.05 | Anti Oxidant |
| Cannabidiol | 0.15 | Active |
| Tocopheryl Acetate | 0.20 | Anti Oxidant |
| Glycerin | 1.00 | Skin Protectant |
| Capric/Caprylic | 1.98 | Emollient |
| Triglyceride | | |
| Brassica Alba Sprout | 0.008 | Active |
| Extract | | |
| Capsaicin | 0.01 | Active |

The first 2 ingredients of the water phase are added to a main vessel and heated to 75° C. The following 8 ingredients are added sequentially to the main vessel and heated to 75° C., while making sure that all waxes and solids are completely dissolved. Glycerin is then added. The mixture is homogenized for 5 min. After homogenization, cooling with moderate mixing begins.

In a separate vessel, an oily liquid comprising the last three ingredients is prepared and added slowly to the main vessel then mixing and cooling is continued to 35° C.

### Example 5

A cream for treatment of diabetes-related indications and having CBD is prepared using the ingredients in table 5:

TABLE 5

| | Amount | |
|---|---|---|
| | (weight/ | |
| Ingredient | weight) % | Function |
| Aqua | 59.15 | Carrier |
| Glycerin | 7.50 | Skin Protectant |
| Caffeine | 0.50 | Active |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 5.60 | Skin Protectant/Carrier |
| Glyceryl Stearate + | 3.00 | Emulsifier |
| Sodium Stearate | | |
| Cetyl Esters | 2.00 | Thickener |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing & calming |
| Steareth-21 | 1.40 | Emulsifier |
| Coco-Caprylate | 2.00 | Light emollient, fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizing & Humectant |
| Aloe Barbadensis Leaf | 0.50 | Soothing and calming |
| Juice (and) Sodium | | |
| Benzoate (and) Potassium | | |
| Sorbate | | |
| Glycerin (and) Water (and) | 0.20 | Soothing and calming |
| Chamomilla Recutita | | |
| (Matricaria) | | |
| Flower Extract (and) | | |
| Sodium Benzoate (and) | | |
| Potassium Sorbate | | |

The first 7 ingredients of the water phase are added to a main vessel and heated to 75° C., making sure that caffeine is dissolved. In a separate vessel the following 19 ingredients of the oily phase are combined and heated to 75° C., while making sure that all waxes and solids are completely dissolved.

The oily phase is added to the water phase in the main vessel at 80° C. and homogenized for 10 min. After homogenization, cooling with moderate mixing begins. At 40° C. a solution of water and hydroxyethyl urea is added while mixing, then the Aloe Barbadensis Leaf Juice and Chamomilla recutita (Matricaria) Flower Extract are added.

### Example 6

A cream for treatment of diabetes-related indications and having CBD is prepared using the ingredients in table 6:

TABLE 6

| | Amount | |
|---|---|---|
| | (weight/ | |
| Ingredient | weight) % | Function |
| Aqua | 63.97 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Methyl Nicotinate | 0.25 | Active |
| Caffeine | 0.035 | Active |
| Dimethicone | 1.00 | Skin Protectant |
| Petrolatum | 3.00 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Leaf | | |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |

The first 8 ingredients of the water phase are added to a main vessel and heated to 85° C. In a separate vessel the following 18 ingredients of the oily phase are combined and heated to 85° C., while making sure that all waxes and solids are completely dissolved.

The oily phase is added to the water phase in the main vessel at 85° C. and homogenized for 10 min. After homogenization, cooling with moderate mixing begins. At 40° C. a solution of water and hydroxyethyl urea is added while mixing, then the Aloe Barbadensis Leaf Juice and Chamomilla recutita (Matricaria) Flower Extract are added and homogenization continues for an additional 2 minutes. Mixing and cooling is continued to 35° C.

### Example 7

A cream for treatment of diabetes-related indications and having CBD is prepared using the ingredients in table 7:

TABLE 7

| | Amount | |
|---|---|---|
| | (weight/ | |
| Ingredient | weight) % | Function |
| Aqua | 63.35 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Methyl Nicotinate | 0.70 | Active |
| Caffeine | 0.200 | Active |
| Dimethicone | 1.00 | Skin Protectant |
| Petrolatum | 3.00 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |

The first 8 ingredients of the water phase are added to a main vessel and heated to 85° C. In a separate vessel the following 18 ingredients of the oily phase are combined and heated to 85° C., while making sure that all waxes and solids are completely dissolved.

The oily phase is added to the water phase in the main vessel at 85° C. and homogenized for 10 min. After homogenization, cooling with moderate mixing begins. At 40° C. a solution of water and hydroxyethyl urea is added while mixing, then the Aloe Barbadensis Leaf Juice and Chamomilla recutita (Matricaria) Flower Extract are added and homogenization continues for an additional 2 minutes. Mixing and cooling is continued to 35° C.

### Example 8

A cream for treatment of diabetes-related indications and having CBD is prepared using the ingredients in table 8:

TABLE 8

| | Amount | |
|---|---|---|
| | (weight/ | |
| Ingredient | weight) % | Function |
| Aqua | 57.25 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 3.00 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Octyldodecanol (and) | 7.00 | Lip plumping agent |
| Quaternium-90 Bentonite | | |
| (and) Propylene (and) | | |
| Carbonate (and) Methyl | | |
| Nicotinate (and) Caffeine | | |
| (and) Niacin | | |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |

The first 6 ingredients of the water phase are added to a main vessel and heated to 85° C. In a separate vessel the following 19 ingredients of the oily phase are combined and heated to 85° C. while making sure that all waxes and solids are completely dissolved.

The oily phase is added to the water phase in the main vessel at 85° C. and homogenized for 10 min. After homogenization, cooling with moderate mixing begins. At 40° C. a solution of water and hydroxyethyl urea is added while mixing, then the Aloe Barbadensis Leaf Juice and Chamomilla Recutita (Matricaria) Flower Extract are added and homogenization continues for an additional 2 minutes. Mixing and cooling is continued to 35° C.

### Example 9

A cream for treatment of diabetes-related indications and having CBD is prepared using the ingredients in table 9:

TABLE 9

| | Amount | |
|---|---|---|
| | (weight/ | |
| Ingredient | weight) % | Function |
| Aqua | 56.65 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 5.60 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Coco-caprylate | 2.00 | Light emollient fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |
| Glycerin, Butylene Glycol, | 3.00 | Active ingredient |
| Water, Hedera Helix (Ivy) | | |
| Leaf/Stem Extract | | |

The first 6 ingredients of the water phase are added to a main vessel and heated to 80° C. In a separate vessel the following 19 ingredients of the oily phase are combined and heated to 80° C., while making sure that all waxes and solids are completely dissolved.

The oily phase is added to the water phase in the main vessel at 80° C. and homogenized for 10 min. After homogenization, cooling with moderate mixing begins. At 40° C. a solution of water and hydroxyethyl urea is added while mixing, then the Aloe Barbadensis Leaf Juice, Chamomilla recutita (Matricaria) Flower Extract and Hedera Helix (Ivy) Leaf/Stem Extract are added and homogenization continues for an additional 2 minutes. Mixing and cooling is continued to 35° C.

### Example 10

A cream for treatment of diabetes-related indications and having CBD is prepared using the ingredients in table 10:

TABLE 10

| | Amount | |
|---|---|---|
| | (weight/ | |
| Ingredient | weight) % | Function |
| Aqua | 56.65 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 5.60 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Coco-caprylate | 2.00 | Light emollient fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | | Skin conditioning |
| Dimonium Chloride | 0.50 | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |
| Butylene Glycol, Centella | 3.00 | Active ingredient |
| Asiatica Extract | | |

The first 6 ingredients of the water phase are added to a main vessel and heated to 80° C. In a separate vessel the following 19 ingredients of the oily phase are combined and heated to 80° C., while making sure that all waxes and solids are completely dissolved.

The oily phase is added to the water phase in the main vessel at 80° C. and homogenized for 10 min. After homogenization, cooling with moderate mixing begins. At 40° C. a solution of water and hydroxyethyl urea is added while mixing, then the Aloe Barbadensis Leaf Juice, Chamomilla Recutita (Matricaria) Flower Extract and Centella Asiatica Extract are added, and homogenization continues for an additional 2 minutes. Mixing and cooling is continued to 35° C.

### Example 11

A cream for treatment of diabetes-related indications and having CBD is prepared using the ingredients in table 11:

TABLE 11

| | Amount | |
|---|---|---|
| | (weight/ | |
| Ingredient | weight) % | Function |
| Aqua | 54.65 | Carrier |
| Glycerin | 7.50 | Skin Protectant |
| Butylene Glycol | 2.00 | Humectant |
| Allantoin | 0.10 | Skin Protectant |
| Methyl Gluceth-20 | 0.50 | Emollient & Humectant |
| Disodium EDTA | 0.05 | Chelating agent |
| Dimethicone | 1.00 | Skin Protectant |
| Petrolatum | 5.60 | Skin Protectant |
| Glyceryl Stearate + | 3.00 | Skin Protectant |
| Sodium Stearate | | |
| Cetyl Esters | 2.00 | Thickener & Emollient |
| Cetyl Alcohol | 1.50 | Skin Protectant |
| Cannabis Sativa Seed Oil | 0.50 | Solvent |
| BHT | 0.05 | Preservative |
| Cannabidiol | 0.15 | Active ingredient |
| Stearyl Alcohol | 1.50 | Thickener & emollient |
| Behentrimonium | 1.00 | Emulsifier |
| Methosulfate (and) Cetyl | | |
| Alcohol (and) Butylene | | |
| Glycol | | |
| Distearyldimonium | 1.00 | Emulsifier |
| Chloride | | |
| Bisabolol | 0.10 | Soothing agent |
| Steareth-21 | 1.40 | Emulsifier |
| Coco-caprylate | 2.00 | Light emollient fast |
| | | spreading |
| Steareth-2 | 0.50 | Emulsifier |
| Phenoxyethanol (and) | 1.00 | Preservative |
| Ethylhexylglycerin | | |
| Polysorbate 60 | 0.50 | Emulsifier |
| Stearamidopropyl PG- | 0.50 | Skin conditioning |
| Dimonium Chloride | | |
| Phosphate (and) Cetyl | | |
| Alcohol | | |
| Tocopheryl Acetate | 0.20 | Anti oxidant |
| Aqua | 2.00 | Carrier |
| Hydroxyethyl Urea | 4.00 | Moisturizer & Umectant |
| Aloe Barbadensis Leaf | 0.50 | Soothing & Calming |
| Juice (and) Sodium | | |
| Benzoate (and) Potassium | | |
| Sorbate | | |
| Glycerin (and) Water (and) | 0.20 | Soothing & Calming |
| Chamomilla Recutita | | |
| (Matricaria) | | |
| Flower Extract (and) | | |
| Sodium Benzoate (and) | | |
| Potassium Sorbate | | |
| Capric/Caprylic | 1.98 | Emollient |
| Triglyceride | | |
| Brassica Alba Sprout | 0.008 | Active ingredient |
| Extract | | |
| Capsaicin | 0.01 | Active ingredient |
| Glycerin, Butylene Glycol, | 3.00 | Active ingredient |
| Water, Hedera Helix (Ivy) | | |
| Leaf/Stem Extract | | |

The first 6 ingredients of the water phase are added to a main vessel and heated to 80° C. In a separate vessel the following 19 ingredients of the oily phase are combined and heated to 80° C., while making sure that all waxes and solids are completely dissolved.

The oily phase is added to the water phase in the main vessel at 80° C. and homogenized for 10 min. After homogenization, cooling with moderate mixing begins. At 40° C. a solution of water and hydroxyethyl urea is added while mixing, then the Aloe Barbadensis Leaf Juice, Chamomilla Recutita (Matricaria) Flower Extract is added. In a separate vessel, the following three ingredients are combined, mixed, then slowly added to the main vessel. Ivy extract is then added, and homogenization continues for an additional 2 minutes. Mixing and cooling is continued to 35° C.

### Example 12

Testing of compositions in diabetic patients.

Diabetic patients, both males and females over 30 years of age are enlisted. A cream prepared according to any of the previous examples is applied to a group of 10 subjects. As control groups, cream without any active ingredient is applied to a group of 5 subjects. A cream with a blood flow enhancer such as Brassica alba sprout extract or capsaicin, but without a cannabinoid is applied to a group of 10 subjects.

The composition is applied to skin of the subject daily, for 28 days. Dermatological evaluation is performed on day 0 and on day 28 of the study by scoring and clinical assessment of the state of the skin. A biometric assessment of skin hydration using MoistureMap MM100® by Enviroderm, United Kingdom, is performed. The apparatus has a capacitance-based sensor that when used on skin, the images of the sensor give graphical information on the near surface hydration distribution and the micro-topography of the tissue.

Biometric assessment of the barrier/lipid function of the skin is performed by determination of the state of transdermal water loss and its restoration of the barrier function at time zero (prior use of the product), 1 hour, 4 hours post administration (immediate action); 24 hours, 14 days and 28 days into the trial. The measurement is performed using Tewameter® by Enviroderm, United Kingdom.

Subjective evaluation of the product by subjects is performed as follows. Subjects fill out a questionnaire at the beginning of the trial (including aspects related to their consumption habits on this type of product). At the end of the trial, the volunteers fill out a questionnaire with information related to the organoleptic perception of the product, the efficacy of the product and future use of the product.

It is expected that the cream applied to the control group will not improve the moisture of the skin of patients. The compositions comprising a cannabinoid and a blood flow enhancing agent are expected to improve parameters measured above to a greater extent than the cream having blood flow enhancing agent alone, without a cannabinoid.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treatment of a patient suffering from a diabetic related condition comprising administering to the patient a composition comprising a cannabinoid, applied via the topical route to the skin of a patient in need thereof.

2. The method according to claim 1 wherein the composition is in the form of a cream, an ointment, an emulsion, a stick, a spray, a roll-on or a moisturizing gel.

3. The method according to claim 1 wherein the cannabinoid is present from between 0.05% to 1% by weight.

4. The method according to claim 3 wherein the cannabinoid is present from between 0.1% to 0.2% by weight.

5. The method according to claim 1 wherein the composition further comprises a blood flow enhancing agent.

6. The method according to claim 5 wherein the blood flow enhancing agent is selected from the group consisting of: Ivy extract, Centella asiatica extract, caffeine, methyl nicotinate, ginger root oil, Brassica alba Sprout Extract, and capsaicin.

7. The method according to claim 6 wherein the blood flow enhancing agent is present in the composition in an amount between 0.001% and 10% by weight.

8. The method according to claim 5, further comprising a skin protectant.

9. The method according to claim 8 wherein the skin protectant is selected from the group consisting of: allantoin, beeswax, calamine, calcium acetate, cetyl alcohol, cocoa butter, colloidal oatmeal, dexpanthenol, dimethicone, glycerin, glyceryl stearate, kaolin, lanolin, mineral oil, petrolatum, zinc acetate, and zinc oxide.

10. The method according to claim 9 wherein the skin protectant is dimethicone.

11. The method according to claim 9 wherein the skin protectant is petrolatum.

12. The method according to claim 9 wherein the skin protectant is glycerin.

13. The method according to claim 1 wherein the cannabinoid is CBD.

14. The method according to claim 1 wherein the condition is selected from the group consisting of: cold feet or hands, numbness in hands or feet, hair loss, dry skin, slow healing of wounds, ulcers, a bacterial infection of the skin, a fungal infection of the skin, itching, acanthosis nigricans, diabetic dermopathy, necrobiosis lipoidica diabeticorum, eruptive xanthomatosis, digital sclerosis, gangrene, necrosis, disseminated granuloma annulare, trans-epidermal water loss, skin redness, skin scales, skin roughness, and pain.

15. The method according to claim 1 wherein the composition is administered to a foot of the patient.

16. The method according to claim 1 wherein the composition is administered between 1 and 4 times daily.

17. The method according to claim 1 wherein the composition is administered in an amount of between 0.1 g to 15 g per administration.

18. A method for enhancing blood flow in a patient suffering from diabetes comprising administering to the patient a composition comprising a cannabinoid, applied via the topical route to the skin.

19. A method according to claim 1 wherein the patient experiences an improvement in skin hydration, skin moisturization, skin redness, soothing and skin smoothness upon administration.

20-21. (canceled)
